(19) United States
(12) Patent Application Publication
(10) Pub. No.: US 2017/0281660 A1
(43) Pub. Date: Oct. 5, 2017

(54) TOPICAL COMPOSITION FOR REDUCING PATHOGEN BINDING

(71) Applicant: GOJO Industries, Inc., Akron, OH (US)

(72) Inventors: Carrie Anne Zapka, Austintown, OH (US); Amanda J. Copeland, Seville, OH (US); Abel Saud, Loveland, OH (US)

(21) Appl. No.: 15/475,938

(22) Filed: Mar. 31, 2017

Related U.S. Application Data
(60) Provisional application No. 62/315,943, filed on Mar. 31, 2016.

(51) Int. Cl. A61K 31/702; A61K 35/747; A61K 38/28

(52) U.S. Cl. CPC A61K 31/702; A61K 38/28; A61K 35/747

(57) ABSTRACT

A method of reducing pathogen binding on skin is provided. The method includes cleaning skin with at least one of a cleanser and a sanitizer and applying a topical composition to the skin. The topical composition is comprised of one or more prebiotic and at least one carrier.

### RELATED APPLICATIONS

This application claims priority to and the benefit of U.S. Provisional Patent Application Ser. No. 62/315,943, entitled “TOPICAL COMPOSITION FOR REDUCING PATHOGEN BINDING” and filed Mar. 31, 2016, the entire disclosure of which is incorporated herein by reference.

### BACKGROUND

Pathogens on the skin are known to cause illness and may be easily transmitted from one person to another. Some pathogens stick strongly to skin. Typically, when pathogens stick to skin, they are more difficult to remove or kill using traditional approaches to skin cleaning and disinfection such as washing with soap or using a waterless sanitizer. Pathogens that are stuck to skin are more dangerous because they remain on the skin longer. The longer the pathogen is on the skin, the more the chance that they will either cause infections on the person with them or be shared with other people.

The overuse of antibiotics is contributing an increase in the types and numbers of antibiotic-resistant pathogens and infections from these pathogens are becoming more dangerous. There is an increasing interest in finding alternative ways to control pathogens without the use of more antimicrobials. Prebiotics and probiotics are being used to control microbes on skin in new ways that do not require the use of antimicrobials. Probiotics are live or inactivated microorganisms that, when either present as part of the normal microbiota or when administered in adequate amounts, confer a health or cosmetic benefit on the host. Benefits from probiotics can be from the microbial components directly or can come from the byproducts of bacterial growth. Prebiotics are non-microbe ingredients that stimulate the growth and/or activity of probiotic microbes or otherwise modify the skin environment to be more favorable for the probiotic microbes in ways that are beneficial to health or provide a cosmetic benefit. Synbiotic is a term used to describe the combined use of a probiotic and a prebiotic for an enhanced benefit.

It is known that some pathogens and beneficial normal (probiotic) skin microbes compete with each other for binding sites on skin. US Patent No. US 2008/0261916 ('916) describes a mixture of prebiotic ingredients used for the prevention, alleviation or treatment of diseases or disorders and that can be administered topically or orally. However, '916 does not decrease the adherence of pathogens on skin or reducing pathogen levels on skin and does not help prevent skin infections or skin-to-skin germ transmission.

### SUMMARY

According to some exemplary embodiments, a composition and method for reducing pathogen binding on a surface is provided. The method includes cleaning a surface with at least one of a cleanser and a sanitizer and applying a topical composition to the surface. The topical composition includes an active ingredient and at least one carrier. The active ingredient includes one or more of a prebiotic material, a probiotic material, and a synbiotic material. The application of the topical composition reduces pathogen binding on the surface by an amount that is statistically significant compared to a placebo or an amount that is at least 5%.

In some exemplary embodiments, the active ingredient includes at least one of a saccharide, oligofructose, polydextrose, sugar alcohol (e.g. xylitol), glucan, mannan, and inulin

In some exemplary embodiments the saccharide is at least one of fructooligosaccharide and galactooligosaccharide.

In some exemplary embodiments, the active ingredient includes a mixture of fructooligosaccharide and inulin.

In some exemplary embodiments, the active ingredient includes a bacterial or a bacterial derivative, such as, for example, Lactobacillus, Streptococcus, Escherichia, Corynebacterium, Lactococcus, Enterococcus, Fusobacterium, Streptomyces, Leuconostoc, Micrococcus, Bifidobacterium, Propionibacterium, Pediococcus, Staphylococcus, and Bacillus.

In some exemplary embodiments, the active ingredient includes a fungal or yeast or a derivative of a fungal or yeast microbe, such as, for example, penicillium, Malassezia, yarrowia, saccharomyces, kluyveromyces, candida, tortulaspora, pichia, debaryomyces, schizosaccharomyces, hansenula, and aspergillus.

In some exemplary embodiments, the active ingredient includes a mixture of α-gluco-oligosaccharide, fructo-oligosaccharide and inactivated Lactobacillus, or a blend of inulin and fructo-oligosaccahride, or Bacillus ferment.

In some exemplary embodiments, the topical composition comprises 0.005 to 10 weight percent active ingredient.

### BRIEF DESCRIPTION OF THE FIGURES

FIG. 1 graphically illustrates the response of Staphylococcus aureus adhesion and invasion potential when treated with a probiotic Bacillus ferment.

FIG. 2 graphically illustrates the effect of prebiotics and probiotics to block binding of an adhesion into epithelial cells by Staphylococcus aureus.

FIG. 3 graphically illustrates the effect of prebiotics and probiotics to block binding of an invasion into epithelial cells by Escherichia coli.

FIG. 4 illustrates the effect of prebiotics and probiotics to block binding of and invasion into epithelial cells by Salmonella typhimurium.

### DETAILED DESCRIPTION

Unless otherwise defined, all technical and scientific terms used herein have the same meaning as commonly understood by one of ordinary skill in the art to which this application pertains. Although other methods and materials similar or equivalent to those described herein may be used in the practice or testing of the exemplary embodiments, exemplary suitable methods and materials are described below. In case of conflict, the present specification including definitions will control. In addition, the materials, methods, and examples are illustrative only and not intended to be limiting of the general inventive concepts.

The terminology as set forth herein is for description of the exemplary embodiments only and should not be construed as limiting the application as a whole. Unless otherwise specified, “a,” “an,” “the,” and “at least one” are used interchangeably. Furthermore, as used in the description of the application and the appended claims, the singular forms “a,” “an,” and “the” are inclusive of their plural forms, unless contradicted by the context surrounding such.

The term “microorganism” or “microbe” as used herein, refers to a tiny organism, such as a virus, protozoan, fungus, or bacterium that can only be seen under a microscope. The collection of microorganisms that live in an environment makes up a microbiota. For example human skin microbiota is all of the microbes on skin or a hospital microbiota would include all of the microbes in a hospital building. The term microbiome is used when referring to the entire habitat, including the microbiota as well as their genomes and the surrounding environment of the microbiota.

The phrase “topical composition” means a composition suitable for application directly to a surface, such as the surface of a human or animal body, including skin, and/or other surfaces, such as hair and nails. The phrase “topical composition” also may refer to a composition suitable for application into a nasal or oral cavity.

The phrase “statistically significant” means p<0.05 for a test composition vs. a control that does not contain the active ingredient. The analysis is completed using 1) a T-test (a statistical examination of two population means) when only comparing one test article vs. one control); or 2) an ANOVA test when comparing two or more test articles vs. controls.

The general inventive concepts relate to a topical composition that contains an active ingredient that includes one or more of probiotics, prebiotics, or a synbiotic mixture thereof, for providing a variety of benefits. In some exemplary embodiments, the topical composition disclosed herein prevents pathogens from adhering to a surface, such as human skin or any inanimate surface. Such adherence prevention includes not only impeding the binding of a pathogen, but also promoting detachment of any already bound pathogen, and otherwise limiting the presence of such pathogens on a surface.

A human's skin microbiota includes resident skin microorganisms that are continuously present on the skin. The resident skin microorganisms are usually non-pathogenic and either commensals (not harmful to their host) or mutualistic (offer a benefit). Resident skin microorganisms are adapted to survive on skin and they eat, reproduce, and excrete, which has an effect on the skin. However, certain transient skin microorganisms may attempt to colonize the skin, which could upset a healthy microbiome. Such transient skin microorganisms may include pathogens, such as pathogenic bacteria, yeasts, viruses, and molds. The particular make-up of a human's microbiome may be different than the make-up of another human's. A resident skin microorganism on one person may be a transient on another.

In some exemplary embodiments, the active ingredient of the topical composition of the present invention includes at least one prebiotic. A prebiotic is any substance or composition that can be utilized as a nutrient by a selected microorganism and can induce the growth and activity of such a microorganism. A prebiotic can also be any substance that modifies the environment of a microbiome to enable the resident or probiotic bacteria to outcompete the transients or pathogens. Non-limiting examples of suitable prebiotics include saccharides, oligofructose, polydextrose, sugar alcohols, glucan, forms of galactan, forms of mannan, and inulin. The particular saccharides may include fructooligosaccharides (FOS) and galactooligosaccharides (GOS). The sugar alcohols may include one or more of lactitol, sorbitol, xylitol, and the like. In some exemplary embodiments, the topical composition includes fructooligosaccharide and inulin. Inulin is a naturally occurring polysaccharide produced by many types of plants, such as the Jerusalem artichoke and chicory. Inulin belongs to a class of dietary fibers known as fructans.

In some exemplary embodiments, the active ingredient of the topical composition includes at least one probiotic. Such probiotics may include, for example, one or more of bacteria, bacteria derivative, yeast, fungal organisms, and byproducts, such as penicillium and yarrowia. Exemplary bacteria include, for example, Lactobacillus, Streptococcus, Escherichia, Lactococcus, Enterococcus, Corynebacterium, Fusobacterium, Streptomyces, Leuconostoc, Micrococcus, Bifidobacterium, Propionibacterium, Pediococcus, Staphylococcus, and Bacillus. Exemplary types of yeast include, for example, saccharomyces, kluyveromyces, candida, tortulaspora, pichia, debaryomyces, schizosaccharomyces, hansenula, and aspergillus.

In some exemplary embodiments, the active ingredient of the topical composition is a synbiotic composition, which is a mixture of prebiotic and probiotic. The prebiotic portion of the synbiotic composition may provide a suitable nutrition source to the probiotic portion, which is believed to increase the likelihood of probiotic survival and colonization or enhance the probiotic benefit.

The topical composition may comprise up to about 20 weight percent of the active ingredient, based upon the total weight of the composition. In some exemplary embodiments, the topical composition comprises about 0.005 to about 10 weight percent of the active ingredient, or from about 0.5 to about 5.0 weight percent of the active ingredient, or from about 1.0 to about 5.0 weight percent of the active ingredient, based upon the total weight of the topical composition.

In some exemplary embodiments, the topical composition further includes a carrier component, such as a base cleaner.

The topical composition may further comprise one or more deposition enhancers. A suitable deposition enhancer works unidirectionally and will allow ingredients within the composition to penetrate deeper into the stratum corneum whilst preventing the loss of materials from the skin. Advantageously, the deposition enhancer provides a cosmetically acceptable skin feel to the formulation.

In one or more embodiments, the deposition enhancers include one or more of surfactants, bile salts and derivatives thereof, chelating agents, and sulphoxides.

Some examples of acceptable deposition enhancers include dimethyl sulphoxides (DMSO), DMA, DMF, 1-dodecylazacycloheptan-2-one (azone), pyrrolidones such as 2-Pyrrolidone (2P) and N-Methyl-2-Pyrrolidone (NMP), long-chain fatty acids such as oleic acid and fatty acids with a saturated alkyl chain length of about C₁₀-C₁₂, essential oils, terpenes, terpenoids, oxazolidinones such as 4-decyloxazolidin-2-one, sodium lauryl sulfate (SLS), sodium laureate, polysorbates, sodium glyacolate, sodium deoxycholate, caprylic acid, EDTA, phospholipids, C₁₂-₁₅ Alkyl Benzoate, pentylene glycol, ethoxydiglycol, polysorbate-polyethylenesorbitan-monolaurate, and lecithin.

In one or more exemplary embodiments, the deposition enhancer is a quaternary ammonium compound such as polyquaternium-6, -7, -10, -22, -37, -39, -74 or -101.

The deposition enhancer may be included in the topical composition in an amount from about 0.005 wt. % to about 10 wt. %, in other embodiments, from about 0.01 wt. % to about 5 wt. %, and in other embodiments, from about 0.05 wt. % to about 3 wt. %, based upon the total weight of the composition.

In one or more exemplary embodiments, the deposition enhancer comprises a hydroxy-terminated polyurethane compound chosen from polyolprepolymer-2, polyolprepolymer-14, and polyolprepolymer-15. Polyolprepolymer-2 is sometimes referred to as PPG-12/SMDI copolymer. The polyurethane compound may be present in the topical composition in an amount from about 0.005 wt. % to about 5 wt. %, in other embodiments, from about 0.01 wt. % to about 3 wt. %, and in other embodiments, from about 0.05 wt. % to about 1 wt. %, based upon the total weight of the composition.

The topical composition may be used as a coating composition for application to any surface, such as, for example, skin, tissue, sink, hair, tabletop, countertop, doorknob, handle, floors, clothing, bed sheets, sinks and countertops in hospitals, food service areas, meat processing plants, and the like. In some exemplary embodiments, the topical composition is used for application to the skin and may be in the form of a skin cleanser, skin sanitizer, skin moisturizer, skin protectant, shampoo, and the like. In some exemplary embodiments, the topical composition comprises one or more of a cleanser, cleaner, sanitizer, a wipe, a lotion, a salve, foam, soap, gel, and a cream. The topical composition may be applied to the skin before, during, or after skin cleaning. In some exemplary embodiments, the topical composition is applied after skin cleaning.

In one or more embodiments, the topical composition includes an alcohol. The alcohol may be a C₁₋₆ alcohol, i.e. an alcohol containing 1 to 6 carbon atoms. Such alcohols may be referred to as lower alkanols. Typically, these alcohols have antimicrobial properties. Examples of lower alkanols include, but are not limited to, methanol, ethanol, propanol, butanol, pentanol, hexanol, and isomers and mixtures thereof In one or more exemplary embodiments, the alcohol comprises ethanol, propanol, or butanol, or isomers or mixtures thereof. In one or more exemplary embodiments, the alcohol comprises isopropanol. In other exemplary embodiments, the alcohol comprises ethanol. In one or more exemplary embodiments, the topical composition comprises a mixture of alcohols. In one or more exemplary embodiments, the topical composition comprises a mixture of ethanol and isopropanol. In one or more exemplary embodiments, the topical composition comprises a mixture of isopropanol and n-propanol.

Generally, the topical composition may comprise at least about 1 percent by weight (wt. %) C₁₋₆ alcohol, based upon the total weight of the composition. In one embodiment, the topical composition comprises at least about 2 weight percent C₁₋₆ alcohol, in another embodiment, the topical composition comprises at least about 10 weight percent C₁₋₆ alcohol, in another embodiment, the topical composition comprises at least about 20 weight percent C₁₋₆ alcohol, in another embodiment, the topical composition comprises at least about 40 weight percent C₁₋₆ alcohol, in another embodiment, the topical composition comprises at least about 50 weight percent C₁₋₆ alcohol, in another embodiment, the topical composition comprises at least about 60 weight percent C₁₋₆ alcohol, in another embodiment, the topical composition comprises at least about 65 weight percent C₁₋₆ alcohol, in yet another embodiment, the topical composition comprises at least about 70 weight percent C₁₋₆ alcohol, and in still yet another embodiment, the topical composition comprises at least about 78 weight percent C₁₋₆ alcohol, based upon the total weight of composition. More or less alcohol may be required in certain instances, depending particularly on other ingredients and/or the amounts thereof employed in the topical composition.

In some exemplary embodiments, the composition includes one or more humectants. Examples of humectants include propylene glycol, hexylene glycol, 1,4-dihydroxyhexane, 1,2,6-hexanetriol, sorbitol, butylene glycol, propanediols, such as methyl propane diol, dipropylene glycol, triethylene glycol, glycerin (glycerol), polyethylene glycols, ethoxydiglycol, polyethylene sorbitol, and combinations thereof. Other humectants include glycolic acid, glycolate salts, lactate salts, urea, hydroxyethyl urea, alpha-hydroxy acids, such as lactic acid, sodium pyrrolidone carboxylic acid, hyaluronic acid, chitin, and the like.

Examples of polyethylene glycol humectants include PEG-4, PEG-6, PEG-7, PEG-8, PEG-9, PEG-10, PEG-12, PEG-14, PEG-16, PEG-18, PEG-20, PEG-32, PEG-33, PEG-40, PEG-45, PEG-55, PEG-60, PEG-75, PEG-80, PEG-90, PEG-100, PEG-135, PEG-150, PEG-180, PEG-200, PEG-220, PEG-240, and PEG-800.

The topical composition may further comprise one or more conditioning or moisturizing esters. Examples of such conditioning or moisturizing esters include cetyl myristate, cetyl myristoleate, and other cetyl esters, diisopropyl sebacate, and isopropyl myristate. The ester may be present in an amount of up to 10% by weight, or from about 0.5 to about 5% by weight, in another embodiment from about 1 to about 2% by weight, based upon the total weight of the topical composition.

In one or more embodiments, the topical composition may include one or more emulsifying agents. Examples of emulsifying agents include stearyl alcohol, sorbitan oleate trideceth-2, poloxamers, and PEG/PPG-20/6 dimethicone. In some exemplary embodiments, the emulsifying agent is present in an amount of up to about 10% by weight, based upon the total weight of the topical composition. In other exemplary embodiments, the emulsifying agent is present in an amount of from about 0.1 to about 5% by weight, or from about 0.5 to about 2% by weight, based upon the total weight of the topical composition.

In one or more embodiments, the topical composition includes one or more miscellaneous skin-conditioners selected from aloe, vitamin E, and C₆₋₁₀ alkane diols.

The topical composition may further comprise a wide range of optional ingredients. The CTFA International Cosmetic Ingredient Dictionary and Handbook, Eleventh Edition 2005, and the 2004 CTFA International Buyer's Guide, both of which are incorporated by reference herein in their entirety, describe a wide variety of non-limiting cosmetic and pharmaceutical ingredients commonly used in the skin care industry, that are suitable for use in the compositions of the present invention. Examples of these functional classes include: abrasives, anti-acne agents, anticaking agents, antioxidants, binders, biological additives, bulking agents, chelating agents, chemical additives; colorants, cosmetic astringents, cosmetic biocides, denaturants, drug astringents, emulsifiers, external analgesics, film formers, fragrance components, opacifying agents, plasticizers, preservatives (sometimes referred to as antimicrobials), propellants, reducing agents, skin bleaching agents, skin-conditioning agents (emollient, miscellaneous, and occlusive), skin protectants, solvents, surfactants, foam boosters, hydrotropes, solubilizing agents, suspending agents (nonsurfactant), sunscreen agents, ultraviolet light absorbers, detackifiers, and viscosity increasing agents (aqueous and nonaqueous). Examples of other functional classes of materials useful herein that are well known to one of ordinary skill in the art include solubilizing agents, sequestrants, keratolytics, topical active ingredients, and the like.

The inventive coating compositions exhibit a pH in the range of from about 2.5 to about 9.0, or a pH in the range of from about 3.5 to about 6, or in the range of from about 4.0 and about 5.5. When necessary, a pH adjusting agent or constituent may be used to provide and/or maintain the pH of a composition.

The form of the composition of the present invention is not particularly limited. In one or more embodiments, topical compositions of the present invention may be formulated as a lotion, a foamable composition, a thickened gel composition, a sprayable liquid, a rinse, or may be applied to a wipe.

In one or more embodiments, the compositions of the present invention may be formulated as a lotion. As is known in the art, lotions include oil-in-water emulsions as well as water-in-oil emulsions, oil-water-oil, and water-oil-water. A wide variety of ingredients may be present in either the oil or water phase of the emulsion. That is, the lotion formulation is not particularly limited.

Examples of lotion formulations include those containing water and/or alcohols and emollients such as hydrocarbon oils and waxes, silicone oils, hyaluronic acid, vegetable, animal or marine fats or oils, glyceride derivatives, fatty acids or fatty acid esters or alcohols or alcohol ethers, lanolin and derivatives, polyhydric alcohols or esters, wax esters, sterols, phospholipids and the like, and generally also emulsifiers (nonionic, cationic or anionic), although some of the emollients inherently possess emulsifying properties.

These same general ingredients may be formulated into a cream rather than a lotion, or into gels, or into solid sticks by utilization of different proportions of the ingredients and/or by inclusion of thickening agents such as gums, carbomers, or other forms of hydrophilic colloids. Very generally, as is known in the art, creams and ointments are typically spreadable in the range from room temperature to skin temperature, and lotions and milks are more flowable within this temperature.

In one or more embodiments, the topical composition of the present invention may be in the form of a thickened gel, with the inclusion of one or more thickeners and optionally one or more stabilizers. Examples of thickeners and stabilizers include hydroxyethyl cellulose hydroxypropyl cellulose, methyl cellulose, carboxymethyl cellulose, and ammonium acryloyldimethyltaurate/VP copolymer. Where the thickener or stabilizer is starch-based, the thickener or stabilizer may be present in an amount of up to about 10% by weight, or in an amount of from about 0.1 to about 5% by weight, or from about 0.2 to about 1% by weight, based upon the total weight of the composition. Where the thickener or stabilizer is a synthetic polymer, the thickener or stabilizer may be present in an amount of up to about 15% by weight, or from about 0.1 to about 10% by weight, or from about 1 to about 2% by weight, based upon the total weight of the composition.

In one or more exemplary embodiments, the topical composition may be thickened with polyacrylate thickeners such as those conventionally available and/or known in the art. Examples of polyacrylate thickeners include carbomers, acrylates/C 10-30 alkyl acrylate cross-polymers, copolymers of acrylic acid and alkyl (C5-C 10) acrylate, copolymers of acrylic acid and maleic anhydride, and mixtures thereof. in one or more embodiments, the gel composition includes an effective amount of a polymeric thickener to adjust the viscosity of the gel to a viscosity range of from about 1000 to about 65,000 centipoise. In one embodiment, the viscosity of the gel is from about 5000 to about 35,000, and in another embodiment, the viscosity is from about 10,000 to about 25,000. The viscosity is measured by a Brookfield RV Viscometer using RV and/or LV Spindles at 22° C.+/−3° C.

As will be appreciated by one of skill in the art, the effective amount of thickener will vary depending upon a number of factors, including the amount of alcohol and other ingredients in the gel composition. In one or more embodiments, an effective amount of thickener is at least about 0.01 wt. %, based upon the total weight of the gel composition. In other embodiments, the effective amount is at least about 0.02 wt. %, or at least about 0.05 wt. %, or at least about 0.1 wt. %. In some exemplary embodiment, the effective amount of thickener is at least about 0.5 wt. %, or at least about 0.75 wt. %, based upon the total weight of the gel. In one or more embodiments, the compositions according to the present invention comprise up to about 10% by weight of the total composition of a polymeric thickener. In certain embodiments, the amount of thickener is from about 0.01 to about 1 wt. %, or from about 0.02 to about 0.4 wt. %, or from about 0.05 to about 0.3 wt. %, based upon the total weight of the antimicrobial gel. The amount of thickener may be from about 0.1 to about 10 wt. %, or from about 0.5% to about 5% by weight, or from about 0.75% to about 2% wt. %, based upon the total weight of the antimicrobial gel.

In one or more embodiments, the gel composition may further comprise a neutralizer. Examples of neutralizing agents include amines, alkanolamines, alkanolamides, inorganic bases, amino acids, including salts, esters and acyl derivatives thereof. Exemplary neutralizing agents include triethanolamine, sodium hydroxide, monoethanolamine and dimethyl stearylamine, Other neutralizing agents are also known, such as HO(CₘH₂ₘ)₂NH, where m has the value of from 2 to 3, and aminomethyl propanol, aminomethyl propanediol, and ethoxylated amines, such as PEG-25 cocamine, polyoxyethylene (5) cocamine (PEG-5 cocamine), polyoxyethylene (25) cocamine (PEG-25 cocamine), polyoxyethylene (5) octadecylamine (PEG-5 stearamine), polyoxyethylene (25) octadecylamine (PEG-25 stearamine), polyoxyethylene (5) tallowamine (PEG-5 tallowamine), polyoxyethylene (15) oleylamine (PEG-15 oleylamine), polyethylene (5) soyamine (PEG-5 soyamine), and polyoxyethylene (25) soyamine (PEG-15 soyamine). A number of these are commercially available under the trade name of Ethomeen® from Akzo Chemie America, Armak Chemicals of Chicago, Ill.

Neutralizers containing sodium hydroxide or sodium hydroxide precursors are the preferred neutralizers for the topical composition of the present invention. Solutions of sodium hydroxide in water are non-limiting examples of neutralizers containing sodium hydroxide.

The neutralizer is employed in an effective amount to neutralize a portion of the carboxyl groups of the thickening agent, and produce the desired pH range. The pH of unneutralized thickening agent dispersed in water is generally acidic. For example, the pH of Carbopol® polymer dispersions is approximately in the range of 2.5 to 3.5, depending upon the polymer concentration. An effective amount of neutralizer, when added to the thickener dispersion, adjusts the pH to a desired range of about 4.1 to 4.8, or of about 4.2 to 4.6. The amount of neutralizer necessary to effect this pH range will vary depending upon factors such as the type of thickening agent, the amount of thickening agent, etc. However, in general, amounts less than 1.0% by weight and preferably ranging from about 0.001 to about 0.3% by weight of the neutralizing agent are considered sufficient and effective.

In one or more embodiments, the topical composition is formulated as a foamable composition. One or more foam agents may optionally be included in the foamable composition.

Any foaming agent conventionally known and used may be employed in the topical composition. In one or more embodiments, the foam agent comprises a non-ionic foam agent such as decyl glucoside or an amphoteric foam agent such as cocamidopropylbetaine. In one or more embodiments, the amount of nonionic or amphoteric foam agent is from about 0.5 to about 3.5 wt. %, in other embodiments from about 1.0 to about 3 wt. %, based upon the total weight of the topical composition. In one or more embodiments, the amount of decyl glucoside or cocamidopropylbetaine is from about 0.5 to about 3.5 wt. %, in other embodiments from about 1 to about 3 wt. %, based upon the total weight of the topical composition.

In some exemplary embodiments, the foaming agents include one or more of silicone glycol and fluorosurfactants. Silicone glycols may be generally characterized by containing one or more Si—O—Si linkages in the polymer backbone. Silicone glycols include organopolysiloxane dimethicone polyols, silicone carbinol fluids, silicone polyethers, alkylmethyl siloxanes, amodimethicones, trisiloxane ethoxylates, dimethiconols, quaternized silicone glycols, polysilicones, silicone crosspolymers, and silicone waxes.

Examples of silicone glycols include dimethicone PEG-7 undecylenate, PEG-10 dimethicone, PEG-8 dimethicone, PEG-12 dimethicone, perfluorononylethyl carboxydecal PEG 10, PEG-20/PPG-23 dimethicone, PEG-11 methyl ether dimethicone, bis-PEG/PPG-20/20 dimethicone, silicone quats, PEG-9 dimethicone, PPG-12 dimethicone, fluoro PEG-8 dimethicone, PEG-23/PPG-6 dimethicone, PEG-20/PPG-23 dimethicone, PEG 17 dimethicone, PEG-5/PPG-3 methicone, bis-PEG-18 methyl ether dimethyl silane, bis-PEG-20 dimethicone, PEG/PPG-20/15 dimethicone copolyol and sulfosuccinate blends, PEG-8 dimethicone\dimmer acid blends, PEG-8 dimethicone\fatty acid blends, PEG-8 dimethicone\cold pressed vegetable oil\polyquaternium blends, random block polymers and mixtures thereof.

The amount of silicone glycol foam agent is not particularly limited, so long as an effective amount to produce foaming is present. In certain embodiments, the effective amount to produce foaming may vary, depending upon the amount of alcohol and other ingredients that are present. In one or more embodiments, the composition includes at least about 0.002 wt. % of silicone glycol foam agent, based upon the total weight of the composition. In another embodiment, the composition includes at least about 0.01 wt. % of silicone glycol foam agent, based upon the total weight of the composition. In yet another embodiment, the composition includes at least about 0.05 wt. % of silicone glycol foam agent, based upon the total weight of the composition.

In some exemplary embodiments, the foam agent is present in an amount of from about 0.002 wt. % to about 4 wt. %, or in an amount of from about 0.01 wt. % to about 2 wt. %, based upon the total weight of the composition. It is envisioned that higher amounts may also be effective to produce foam. All such weights as they pertain to listed ingredients are based on the active level, and therefore, do not include carriers or by-products that may be included in commercially available materials, unless otherwise specified.

In other embodiments, it may be desirable to use higher amounts of foam agent. For example, in certain embodiments where the foaming composition of the present invention includes a cleansing or sanitizing product that is applied to a surface and then rinsed off higher amounts of foam agent may be employed. In these embodiments, the amount of foam agent is present in amounts up to about 35 wt. %, based upon the total weight of the composition.

The topical composition of the present invention may be formulated as an aerosol or non-aerosol foamable composition,

In one or more embodiments, the viscosity of the non-aerosol foamable composition is less than about 100 mPas, in one embodiment less than about 50 mPas, and in another embodiment less than about 25 mPas.

The composition of the present invention may be employed in any type of dispenser typically used for gel products, for example pump dispensers. A wide variety of pump dispensers are suitable. Pump dispensers may be affixed to bottles or other free-standing containers. Pump dispensers may be incorporated into wall-mounted dispensers. Pump dispensers may be activated manually by hand or foot pump, or may be automatically activated. Useful dispensers include those available from GOJO Industries under the designations NXT® and TFX™ as well as traditional bag-in-box dispensers. Examples of dispensers are described in U.S. Pat. Nos. 5,265,772, 5,944,227, 6,877,642, 7,028,861, 7,611,030, and 7,621,426, all of which are incorporated herein by reference. In one or more embodiments, the dispenser includes an outlet such as a nozzle, through which the composition is dispensed. In some exemplary embodiments, the topical composition is used in dispensers that employ foaming pumps, which combine ambient air or an inert gas and the composition in a mixing chamber and pass the mixture through a mesh screen.

In one or more embodiments, the topical composition is integrated into wipe composition. Wipe compositions in accordance with this invention include at least one alcohol, a C₁₋₁₀ alkanediol enhancer, and are applied to a wipe substrate. In some exemplary embodiments, the wipe composition is alcohol-free.

Wipe substrates used in antimicrobial wipes are further described in U.S. Pat. Nos. 5,686,088, 6,410,499, 6,436,892, 6,495,508, 6,844,308. In one or more embodiments, the wipe may comprise a laminate formed by spunbonding/meltblowing/spunbonding (SMS). Generally, an SMS material contains a meltblown web sandwiched between two exteriors spunbond webs. SMS materials are further described in U.S. Pat. Nos. 4,041,203, 5,169,706, 5,464,688, and 4,766,029, and are commercially available, for example from Kimberly-Clark Corporation under marks such as Spunguard 7 and Evolution 7. The SMS laminate may be treated or untreated.

In some exemplary embodiments, the topical composition increases the presence of resident bacteria, while decreasing the presence of transient bacteria, such as skin pathogens, by inhibiting or interfering with the binding of skin pathogens onto a skin surface. The prebiotics and/or or probiotics contained in the inventive topical composition show the ability to differentially affect the binding and survival of normal skin bacteria, such as Staphylococcus epidermis vs. skin pathogens, such as Staphylococcus aureus. For instance, the topical composition can prevent pathogens from binding to the skin in one of multiple ways which may involve one or more of either specific targeted chemical binding and/or non-specific physical steric interference. The composition may specifically block the binding site of the pathogen which interrupts the microbe's ability to bind to skin. The composition may also block the microbe binding locations on the treated surface. For example, the composition may bind to areas of skin cells that pathogens stick to thus reducing the area that pathogens can bind to thus reducing the amount of pathogens present on skin. For example, the topical composition could bind to fibronectin, fibronectin-binding protein, fibrinogen, adhesion molecules, laminins, extracellular matrix proteins, receptors, or other sites known to be involved in binding of pathogens to human tissue or inanimate surfaces. The composition may also reduce pathogen binding by steric interference such as by providing a barrier between the pathogen and binding surface that limits the pathogen's ability to get close enough to the surface binding site to form a bond. Interfering with binding of pathogens on skin reduces the level of pathogens that are able to adhere to the skin, the risk of infection and transmission of the pathogen is also reduced.

In some exemplary embodiments, the topical composition will decrease the binding of one or more of pathogens known to cause illness or infection among humans in hospitals, food processing, food service, healthcare, education and the like. Exemplary pathogens that may be reduced include all organisms considered pathogenic by public health bodies such as NIH, CDC, FDA and the like. Further non-limiting examples include any microbe referred to in the FDA Bad Bug Book (http://www.fda.gov/downloads/Food/FoodborneIllnessContaminants/UCM297627.pdf) such as E. coli, Salmonella, Camplylobacter, Shigella, etc. Other non-limiting examples include those listed as pathogens by the active bacterial core surveillance division of the CDC (http ://www. cdc.gov/abcs/pathogens/pathogen-links.html) such as MRSA, Streptococcus pyogenes, Haemophilus influenza, Legionella, etc. Further non-limiting examples includes those on the NIH emerging infectious diseases and pathogens list (http ://www.niaid.nih.gov/topics/biodefenserelated/biodefense/pages/cata.aspx), which includes microbes that cause anthrax, plague, botulism, smallpox, Ebola, etc. The following examples are included for purposes of illustration and are not intended to limit the scope of the methods described herein.

### EXAMPLES

The effect of exemplary topical compositions was investigated for pathogen blocking potential. Three pathogenic bacterial strains were tested: Methicillin resistant Staphylococcus aureus strain Mu50 ATCC 33591, Escherichia coli strain K12, Salmonella enterica serovar typhimurium strain 14028S. Each strain was tested against the following exemplary topical compounds: DMEM (cell culture medium, control), 100 nM dexamethasone (DEX, control steroidal anti-inflammatory), 0-5% Ecoskin (α-gluco-oligosaccharide, fructo-oligosaccharide and inactivated Lactobacillus), 0-5% Bacillus ferment, and 0-5% of a prebiotic blend of inulin and fructo-oligosaccahride.

Differentiated colonic epithelial cells were treated with the topical compounds and a bacterial strain (Staphylococcus aureus, Staphylococcus epidermidis, Escherichia coli, or Salmonella typhimurium) was then added individually. Each microbe was grown to the mid-log phase in an acceptable medium and the concentration adjusted so that the amount of bacteria added to the wells was approximately 100 microbes per well (in a 96 well tray with total volume of 100 uL). The cells were then incubated with each bacterial strain for one hour. A Gentamicin protection assay was used to determine adhered and invaded bacteria. Polymerase chain reaction (PCR) using 16S gene primers was used to determine the number of adhered bacteria, as well as the number of bacteria that invaded into the host cells.

Staphylococcus aureus

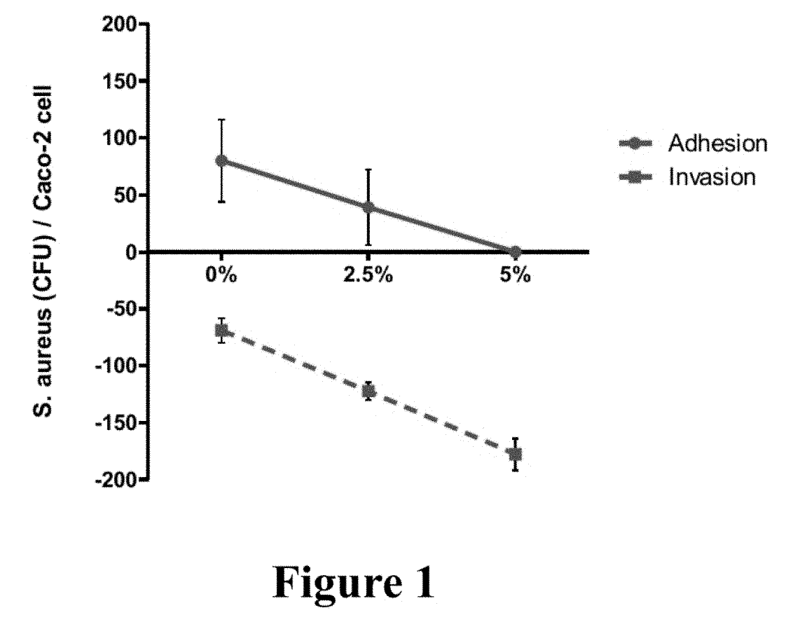

FIG. 1 illustrates the dose-dependent response of Staphylococcus aureus adhesion and invasion potential. Bacillus ferment had a consistent increase in the dose response. Particularly, 5% Bacillus ferment resulted in the lowest adhesion occurrence overall.

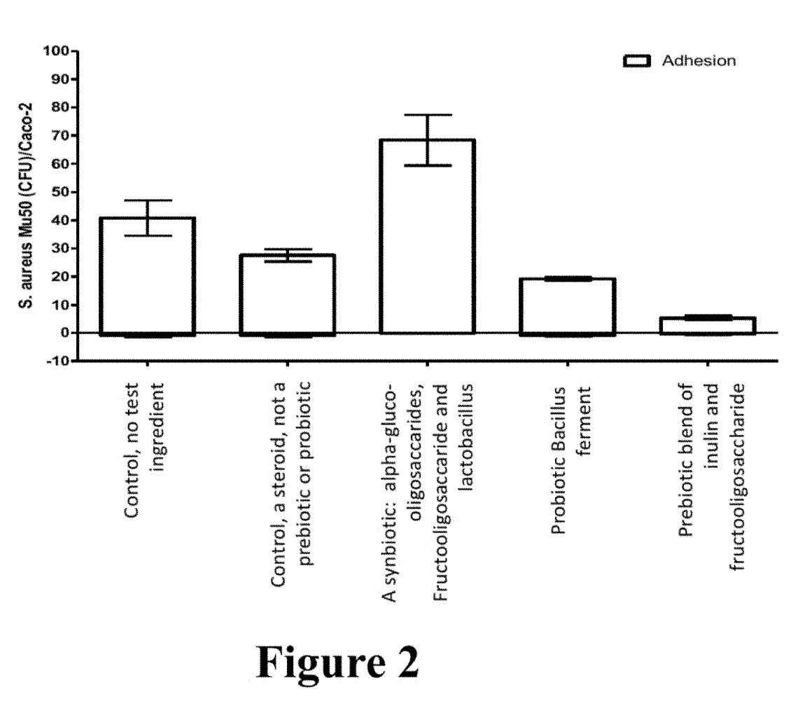

FIG. 2 illustrates the response of Staphylococcus aureus when treated with each of the exemplary topical compounds, each at 5%. As illustrated, treatment with a prebiotic blend of inulin and fructo-oligosaccharide lowered the presence of adhesion, as compared to the untreated control. The probiotic Bacillus ferment also reduced the adhesion.

Escherichia coli

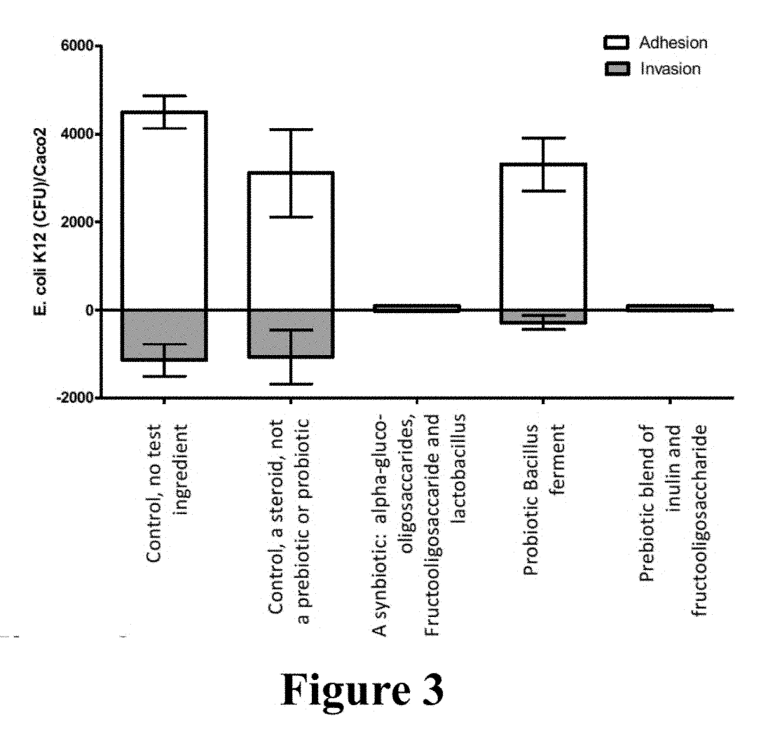

FIG. 3 illustrates the response of Escherichia coli when treated with each of the exemplary topical compounds at 5%. As illustrated, the control resulted in a high occurrence of adhesion and cell invasion, as compared to the treated cells. In contrast, treatment with either the synbiotic Ecoskin or the prebiotic blend of inulin and fructo-oligosaccharide greatly reduced both adhesion of the bacteria and invasion to the point that such was virtually eliminated.

Salmonella typhimurium

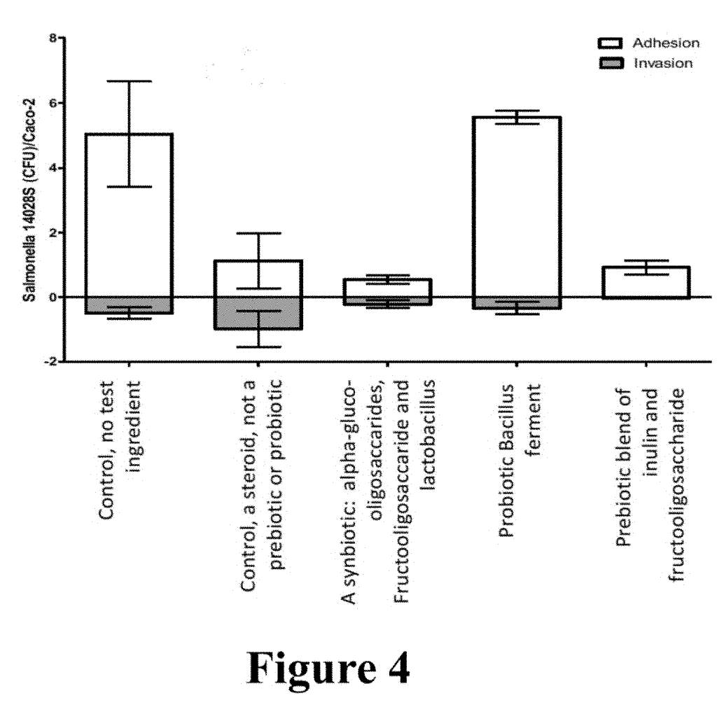

FIG. 4 illustrates the response of S. typhimurium when treated with each of the topical compounds at 5%. As illustrated, the control resulted in an occurrence of adhesion and cell invasion, as compared to most of the treated cells. In contrast, treatment with either the synbiotic Ecoskin or the prebiotic blend of inulin and fructo-oligosaccharide greatly reduced both adhesion of the bacteria and invasion to the point that such was virtually eliminated.

Based on the above-described results, the blocking compounds were found to demonstrate microbe-specific blocking patterns. Probiotic, prebiotic, and synbiotic compositions were each able to reduce pathogen adhesion and/or invasion in at least one pathogen.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A topical composition for reducing pathogen binding on a surface, said composition comprising:
an active ingredient; and
at least one carrier, wherein said active ingredient comprises one or more of a prebiotic material, a probiotic material, and a synbiotic material, wherein said topical composition reduces pathogen binding on a surface by a statistically significant amount.

2. The topical composition of claim 1, wherein said one or more prebiotic includes at least one of a saccharide, oligofructose, polydextrose, sugar alcohol, glucan, mannan, and inulin.

3. The topical composition of claim 2, wherein said saccharide is at least one of fructooligosaccharide and galactooligosaccharide.

4. The topical composition of claim 1, wherein said active ingredient includes a mixture of fructooligosaccharide and inulin.

5. The topical composition of claim 1, wherein said probiotic material includes one or more of a bacteria, a bacteria derivative, a yeast, and a fungus.

6. The topical composition of claim 5, wherein said bacteria includes one or more of Lactobacillus, Streptococcus, Escherichia, Corynebacteria, Lactococcus, Enterococcus, Fusobacterium, Streptomyces, Leuconostoc, Micrococcus, Bifidobacterium, Propionibacterium, Pediococcus, Staphylococcus, and Bacillus.

7. The topical composition of claim 1, wherein said active ingredient comprises α-gluco-oligosaccharide, fructo-oligosaccharide and inactivated Lactobacillus.

8. The topical composition of claim 1, wherein said active ingredient if Bacillus ferment.

9. The topical composition of claim 1, wherein said carrier includes one or more of a base cleanser, a sanitizer, serum, and lotion.

10. The topical composition of claim 1, wherein said surface is skin.

11. The topical composition of claim 1, wherein said topical composition comprises 0.005 to 10 weight percent active ingredient.

12. The topical composition of claim 1, wherein said topical composition comprises about 1.0 to 5.0 weight percent active ingredient.

13. The topical composition of claim 1, wherein the application of said topical composition reduces pathogen binding on a surface by at least 5%.

14. The topical composition of claim 1, wherein the application of said topical composition reduces pathogen binding on a surface by at least 10%.

15. A method of reducing pathogen binding on a surface, said method comprising the steps of:
cleaning a surface with at least one of a cleanser and a sanitizer; and
applying a topical composition to the surface, wherein said topical composition is comprised of an active ingredient and at least one carrier, said active ingredient comprising one or more of a prebiotic material, a probiotic material, and a synbiotic material, wherein the application of said topical composition reduces pathogen binding on a surface by a statistically significant amount.

16. The method of claim 15, wherein said surface is skin.

17. The method of claim 15, wherein said prebiotic material includes at least one of a saccharide, oligofructose, polydextrose, sugar alcohol, glucan, mannan, and inulin.

18. The method of claim 17, wherein said saccharide is at least one of fructooligosaccharide and galactooligosaccharide.

19. The method of claim 15, wherein said topical composition include a mixture of fructooligosaccharide and inulin.

20. The method of claim 15, wherein said probiotic material includes one or more of bacteria, bacteria derivative, yeast, and fungus.

21. The method of claim 20, wherein said bacteria includes one or more of Lactobacillus, Streptococcus, Escherichia, Lactococcus, Enterococcus, Corynebacterium, Fusobacterium, Streptomyces, Leuconostoc, Micrococcus, Bifidobacterium, Propionibacterium, Pediococcus, Staphylococcus, and Bacillus.

22. The method of claim 15, wherein said active ingredient comprises α-gluco-oligosaccharide, fructo-oligosaccharide and inactivated Lactobacillus.

23. The method of claim 15, wherein said active ingredient comprises a blend of inulin and fructo-oligosaccharide.

24. The method of claim 15, wherein said active ingredient if Bacillus ferment.

25. The method of claim 15, wherein said carrier includes one or more of a base cleanser, a sanitizer, serum, and lotion.

26. The method of claim 25, wherein said carrier is a sanitizer and has an alcohol content between 50 and 80 wt %.

27. The method of claim 15, wherein said topical composition has a pH between 2.5 and 9.0.

28. The method of claim 15, wherein said topical composition comprises 0.005 to 10 weight % active ingredient.

29. The method of claim 28, wherein said topical composition comprises about 1.0 to 5.0 weight % active ingredient.

30. The method of claim 15, wherein the application of said topical composition reduces pathogen binding on a surface by at least 5%.

31. The method of claim 15, wherein the application of said topical composition reduces pathogen binding on a surface by at least 10%.
